(19) United States
(12) Patent Application Publication
(10) Pub. No.: US 2009/0137997 A1
(43) Pub. Date: May 28, 2009

(54) Interventional system providing tissue ablation cooling

(75) Inventor: Hongxuan Zhang, Schaumburg, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/246,989

(22) Filed: Oct. 7, 2008

Related U.S. Application Data
(60) Provisional application No. 60/989,226, filed on Nov. 20, 2007.

(51) Int. Cl. A61B 18/24; A61B 18/18

(52) U.S. Cl. 606/11; 606/33

(57) ABSTRACT

An interventional system integrates a laser optical cooling unit into a medical catheter device for treating coronary artery disease (CAD) to safely steer and control cardiac tissue temperature and thermal patterns to prevent cardiac disease, tissue damage and myocardial ischemia or infarction, for example. An interventional system provides tissue ablation and cooling using a catheterization device. The catheterization device for internal anatomical insertion includes, a laser light emitting node for optical cooling of anatomical tissue, an ablation node for use in surgical removal of anatomical tissue and a temperature sensor. The temperature sensor senses temperature of anatomical tissue for use in regulating heating and cooling of tissue resulting from use of the ablation node and the laser light emitting node.

This is a non-provisional application of provisional application Ser. No. 60/989,226 filed Nov. 20, 2007, by H. Zhang.

### FIELD OF THE INVENTION

This invention concerns an interventional system providing optical cooling of anatomical tissue and an ablation node for use in surgical removal of anatomical tissue.

### BACKGROUND OF THE INVENTION

Coronary Artery Disease (CAD) and heart-related problems are responsible for a substantial proportion of patient fatalities. The principal manifestations of CAD are coronary artherosclerosis (hardening of the coronary arteries) or stenosis (narrowing of the arteries), both of which ultimately force a reduction in the coronary circulation (myocardial ischemia). An ischemic episode (either due to severe narrowing, or artery blockage) generally leads to angina pectoris, or a heart attack. During ischemia, various portions of heart muscle receive less oxygen which can ultimately lead to irreversible scarring and necrosis of the muscle tissue (myocardial infarction), reducing the efficiency with which the heart can pump blood to the rest of the body and possibly leading to fatal cardiac arrhythmias. Recent research demonstrates that change in epicardial temperature is a useful indicator of myocardial ischemia, and that cardiothermography can assess the degree and extent of myocardial ischemia. Furthermore, the intra-cardiac tissue temperature and thermal pattern management may support protection of the heart tissue and prevent any potential disease or medical treatment related tissue injury.

Medical treatment of cardiac disease also requires temperature controlling and management. For example, Cardiac ablation is an important invasive treatment used to treat many types of heart arrhythmia, which includes atrial fibrillation, atrial flutter, etc. Typically, an RF energy ablation catheter is used in an electrophysiology (EP) laboratory to treat patient arrhythmia by destroying pathological conduction tissue and thus returning the patient to a healthy rhythm and/or preventing a patient from going into an unhealthy heart rhythm. RF energy is directed through the EP catheter to small areas of the heart muscle that are causing and/or conducting the abnormal heart rhythm. This energy “disconnects” the underlying aberrant pathway of the abnormal cardiac rhythm. In some cases, there is a need to disconnect the electrical pathway between the upper chambers (atria) and the lower chambers (ventricles) of the heart (and insert a pacemaker) and ablation energy is used for this as well. Typically a liquid reperfusion (pumping) procedure is employed for tissue temperature cooling in the cardiac ablation treatment. This adds to medical treatment complexity and may potentially cause additional catheter and heart tissue problems, such as thrombosis, infection, sepsis and necrosis, for example.

In known tissue ablation systems, temperature control and thermal pattern steering has limited efficiency and reliability. The complexity of external liquid pumping functions used by known ablation systems adds cost, reduces reliability and maintainability and potentially increases risk to myocardial tissue and cardiac function. Known intra-cardiac catheters, such as RF catheters, typically have one single point for tissue cooling and temperature control which reduces system flexibility. A system according to invention principles addresses these deficiencies and related problems.

### SUMMARY OF THE INVENTION

An interventional system employs an optical laser cooling unit for use in angioplasty, cardiac tissue treatment and other medical procedures and in one embodiment integrates the laser optical cooling unit into a medical catheter device for treating coronary artery disease (CAD) to safely steer and control cardiac tissue temperature and thermal patterns to prevent cardiac disease, tissue damage and myocardial ischemia or infarction, for example. An interventional system provides tissue ablation and cooling using a catheterization device. The catheterization device for internal anatomical insertion includes, a laser light emitting node for optical cooling of anatomical tissue, an ablation node for use in surgical removal of anatomical tissue and a temperature sensor. The temperature sensor senses temperature of anatomical tissue for use in regulating heating and cooling of tissue resulting from use of the ablation node and the laser light emitting node.

### BRIEF DESCRIPTION OF THE DRAWING

FIG. 1 shows an interventional system comprising an RF ablation catheter providing tissue ablation and cooling, according to invention principles.

FIG. 2 illustrates an angioplasty procedure performed using an interventional system comprising an RF ablation catheter providing tissue ablation and cooling, according to invention principles.

FIG. 3 illustrates application of a catheter integrating RF ablation o and optical cooling units and supporting electrophysiology (EP) signal monitoring, according to invention principles.

FIG. 4 shows an electro-cautery system, according to invention principles.

FIG. 5 shows an external anatomical tissue cooling system, according to invention principles.

### DETAILED DESCRIPTION OF THE INVENTION

An interventional system provides tissue ablation and optical cooling in a catheterization device, in one embodiment, for cardiac tissue temperature and thermal pattern steering and control. The catheter is advantageously usable for, intra-cardiac ablation energy removal (for cardiac tissue overheating, burning or necrosis), for tissue treatment to prevent unwanted arrhythmias, side effects of the medical procedures, and cardiac tissue post-procedure treatment. The catheter is usable to reduce potential ischemia or infarction risks during intra-cardiac artery vessel endarterectomy and stent installation and improve performance and safety of current intra-cardiac medical procedures. The catheter optical cooling system is used in a closed thermo-regulatory loop for heart tissue safety and cardiac function applications employing continuous temperature and thermal pattern monitoring and mapping.

The optical laser based cooling unit provides an easier to use, more reliable, safer cooling system for cardiac tissue treatment. Optical laser cooling advantageously eliminates a need to inject a liquid or fluid on a heart or patient anatomy thereby reducing risk and complexity of a medical treatment procedure. The optical laser based cooling unit may be integrated into any kind of catheter, such as an EP catheter, ablation catheter or balloon (angioplasty) catheter, for example. The optical laser based cooling unit may employ small size fibers to transmit optical laser light, absorb intra-cardiac heat and control tissue temperature. Optical laser cooling is usable to extract ablation heat in a cardiac arrhythmia ablation procedure and may be used independently to treat myocardial tissue and to prevent potential arrhythmias, such as myocardial ischemia and infarction. The system in other embodiments provides multi-point (e.g., multi-node) cooling and may focus on a small local region for precise cardiac tissue treatment and cost efficiency. Further, the optical laser cooling unit reduces spurious electrical signal noise and improves signal quality of intra-cardiac EP monitoring and recording.

Temperature is an indicator of function status of human tissue and temperature control of heart tissue is used for patient safety. Lowered temperature is used to prevent irreversible scarring and necrosis of heart muscle tissue during myocardial ischemia (MI) and infarction, for example. In comparison, known systems typically use external liquid pumping, electrical or magnetic cooling methods with attendant reduced reliability, electrical noise and increased risk. Known liquid reperfusion (pumped) procedures employed in tissue cooling in cardiac treatment involve medical treatment complexity and potentially cause additional problems and risks to heart tissue, such as thrombosis, sepsis and necrosis, for example. Known liquid pumped cooling systems require preparation of a supply of an external liquid. Other known electrical cooling methods (such as thermoelectric coolers, TECs) may generate electrical noise or artifacts which may potentially decrease the signal to noise ratio for intra-cardiac electrophysiological activities and signal recording and monitoring.

A processor as used herein is a device for executing stored machine-readable instructions for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example. A processor may be electrically coupled with any other processor enabling interaction and/or communication there-between. A processor may also be electrically coupled with any other processor, or be electrically coupled by being within stored executable instruction enabling interaction and/or communication with executable instructions comprising another processor. A user interface processor or generator is a known element comprising electronic circuitry or software or a combination of both for generating display images or portions thereof. A user interface comprises one or more display images enabling user interaction with a processor or other device.

An executable application comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity. An object or data object comprises a grouping of data, executable instructions or a combination of both or an executable procedure.

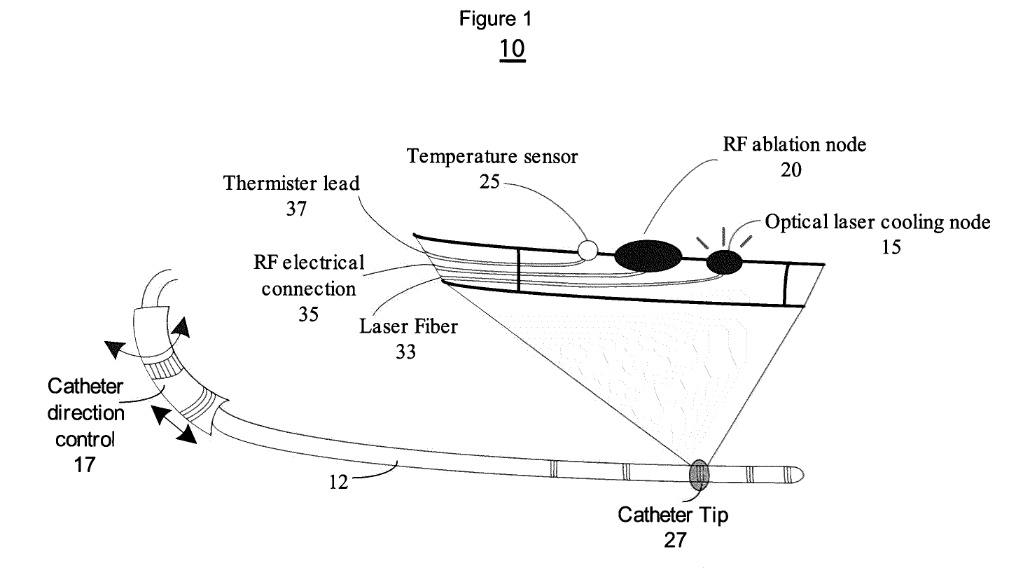

FIG. 1 shows interventional system 10 comprising an RF ablation catheter providing tissue ablation and cooling. Interventional system 10 comprises catheter 12 having directional and maneuvering controls 17 at one end and a unit 27 located towards the other end of the catheter including cooling node 15, ablation node 20 and temperature sensor 25. Interventional system 10 includes optical laser cooling node 15 for emitting laser light provided via fiber 33 for cooling anatomical tissue. RF ablation node 20 powered via electrical connection 35 emits RF radiation energy used for thermal destruction of tissue. Temperature sensor (e.g., thermister) 25 continuously monitors temperature and is electrically connected to a control unit (not shown to preserve drawing clarity) via electrical connection 37 to provide temperature representative data to the control unit. In atrial fibrillation (cardiac arrhythmia) treatment, for example, RF ablation energy (20-50 J) is delivered via node 20 to atrial tissue which temporarily increases local tissue temperature. Temperature sensor 25 is near the ablation point and continuously monitors temperature and provides temperature indicative data via lead 37 used by the control unit to determine thermal patterns and as feedback to control cooling via optical laser cooling node 15 located near the ablation point to prevent tissue overheating. The optical laser cooling unit is also used in different kinds of ablation procedure and cardiac tissue treatment to prevent heart tissue overheating, burning and unexpected cardiac arrhythmias. Further, in other embodiments, system 10 may have multiple ablation nodes, multiple temperature sensors (one thermal sensor for each ablation lead point) and multiple optical laser cooling nodes corresponding to individual ablation nodes. In another embodiment, the RF ablation node is replaced with a laser ablation node for use in surgical removal of anatomical tissue.

Optical laser cooling node 15 cools heart tissue by making light bounce off the tissue. This occurs because light bounces off the tissue with more energy than when it hits the cardiac tissue. The cooling optical laser light comprises a stream of photons with no mass. The laser light photons are like ping-pong balls compared to a bowling ball when they are bouncing on the atoms of cardiac tissue. The cardiac tissue atoms are pushed around by bouncing laser light off them. By adjusting the laser power and laser position, the atom movement of the cardiac tissue may be slowed down. Consequently the temperature of the tissue is reduced. The optical cooling involves preparing an ensemble of paramagnetic atoms in an external magnetic field in a lowest Zeeman sublevel by optical pumping, waiting for collision-induced thermal repopulation of the higher sublevels (which proceeds at the expense of kinetic energy of the atoms) and optically pumping the atoms back into the lowest Zeeman sublevel, thus cooling the spin system because the emitted photons show higher energy than those absorbed. The optical laser cooling prevents potential cardiac arrhythmia or side effects, especially in some angioplasty procedures, such as ischemia happening during PTCA (Pecutaneous Transluminal Coronary Angioplasty) procedure, for example. The optical laser cooling is also used to treat and cure (terminate) existing cardiac pathology and tissue malfunction irregularity with cooling or freezing.

During ablation medical treatment, such as the treatment of atrial fibrillation, RF energy (20-50 J, 30-90 seconds) is delivered to an atrial chamber, for example. There is a risk that accumulated heat may injure atrial tissue if the temperature of ablated tissue is not controlled. Optical laser cooling node 15 efficiently absorbs heat and prevents adverse thermal effects from the ablation procedure including related secondary injury or necrosis. Before, during and after the ablation procedure, the cardiac tissue temperature is continuously monitored by temperature sensor 25 and optical laser cooling is applied at programmed times controlled and selected by the control unit. Optical laser cooling node 15 is activated and controlled in real-time by one or more of multiple control signals including a pulsed or continuous signal, a predetermined time interval signal, and a power level signal.

Use of optical cooling node 15 prevents overheating or burning during cardiac arrhythmia ablation comprising invasive treatment used to treat many types of heart arrhythmia, which includes atrial fibrillation, atrial flutter, AV re-entrant tachycardia, accessory pathway arrhythmias (such as WPW, Wolff-Parkinson-White) and ventricular tachycardia. Typically, an RF energy ablation catheter is used in an electrophysiology (EP) laboratory to treat patient arrhythmia by destroying pathological conduction tissue and thus returning the patient to a healthy rhythm and/or preventing a patient from going into an unhealthy heart rhythm. The RF energy is directed through the EP catheter to small areas of the heart muscle that are causing and/or conducting the abnormal heart rhythm. The RF energy is used to disconnect the underlying aberrant pathway of the abnormal cardiac rhythm. The delivered energy and related thermal effects of the cardiac arrhythmia ablation may accumulate within the ablated tissue location. The resulting temperature of the ablation tissue may increase which may result in cardiac tissue injury, necrosis, and unwanted potential side effects including cardiac arrhythmias and secondary heart lesion.

The optical laser cooling system is also usable as an independent medical method for cardiac arrhythmia treatment and heart tissue/function rehabilitation. For example, during some angioplasty procedures, such as a PTCA (Pecutaneous Transluminal Coronary Angioplasty) procedure, when a balloon is inflated, the blood flow in the descending branches of the corresponding coronary (like LAD or RCA) vessels from the balloon are blocked and this may result in myocardial ischemia or infarction of the cardiac tissue which the descending branches of the coronary artery are supporting. Optical laser cooling may be used to reduce temperature to control and steer the function of the cardiac tissue and prevent myocardial ischemia procedure.

In one embodiment, the system 10 structure involves an ablation catheter providing optical laser ablation, optical laser cooling and substantially real-time, continuous temperature measurement and feedback control. The temperature measurement and feedback is controllable and adjustable to further reduce unwanted side effects, burning heat risk from the RF ablation in cardiac tissue. The optical cooling system accurately controls heat and temperature of heart tissue, for example, and does not add risk to the medical application and treatment. In other embodiments, the optical laser cooling system is also advantageously used in an electro-cautery device and in a device for external anatomical cooling.

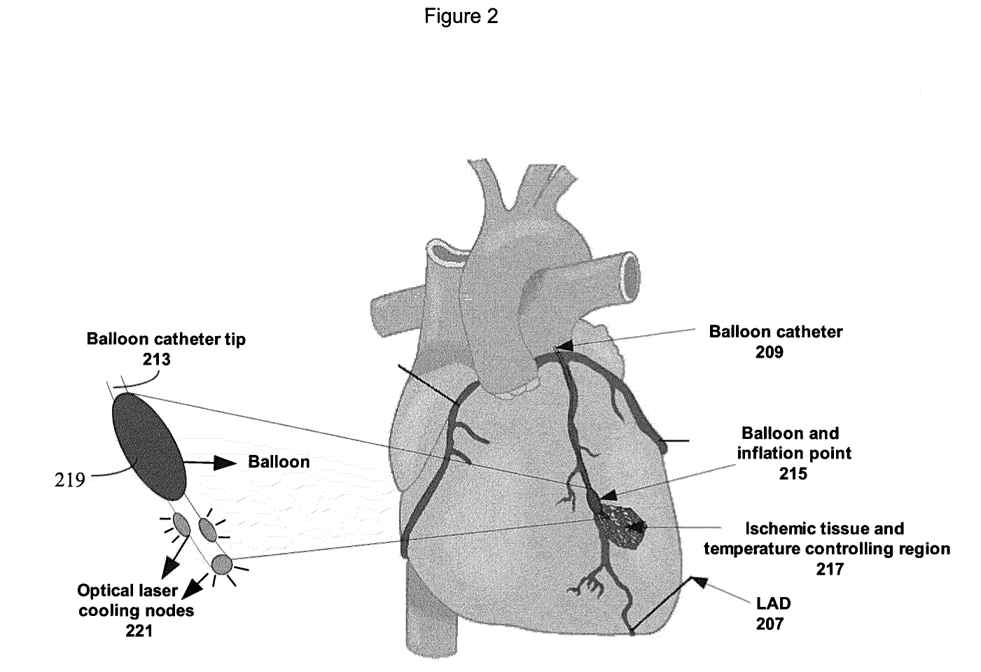

FIG. 2 illustrates an angioplasty procedure performed using balloon catheter 209 having tip 213 including multiple laser optical cooling nodes 221 and balloon 219 for performing a PTCA procedure, specifically a rotational atherectomy procedure. The procedure involves cleaning and installing a stent in the LAD artery vessel 207 to terminate ischemia risk. During balloon insertion and balloon inflation, near the second branch of descending left artery 215 there is an emerging low blood flow region and potentially ischemia. Optical laser cooling from tip 213 of balloon catheter 209 is applied to ischemic tissue in region 217 to reduce ischemia related risk and protect myocardial tissue. Optical laser cooling nodes 221 are usable in electrophysiological procedures including PTCA (rotational atherectomy), intra-cardiac medical treatment, such as Angiojet, DCA (directional coronary atherectomy) and endarterectomy procedures, for example.

During the PTCA treatment, balloon 219 is inflated for 30 to 90 seconds, for example, which means the rest of branches of the coronary artery are blocked for a period of time. This potentially causes myocardial ischemia or infarction which can permanently injure the tissue. The balloon angioplasty catheter tip 213 including multiple optical laser cooling nodes 221, advantageously decrease temperature and create a suitable thermal environment for potential ischemic region 217 and reduces risk of the side-effects in the PTCA treatment procedure.

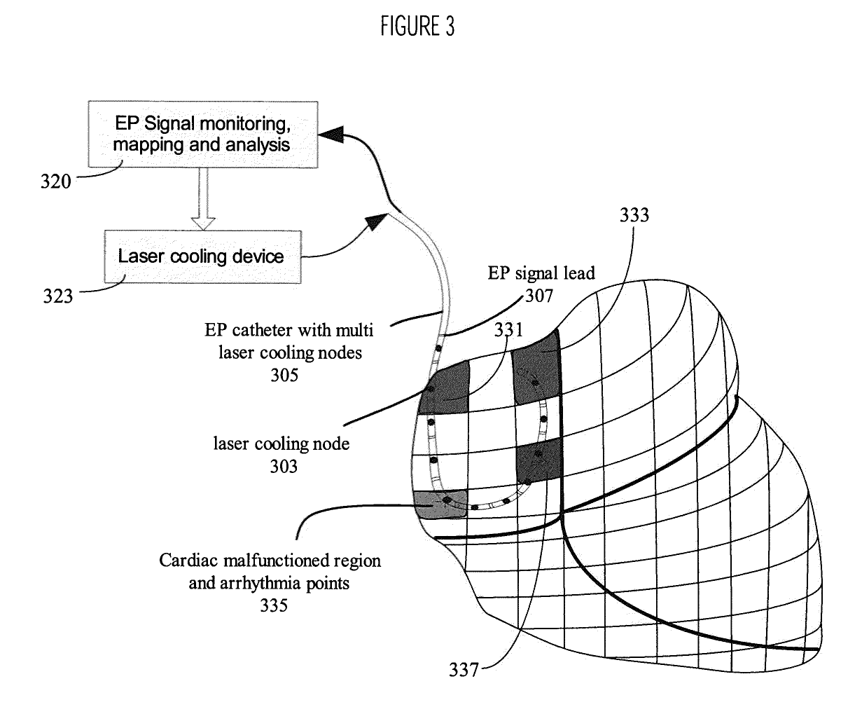

FIG. 3 illustrates application of catheter 305 integrating RF ablation and optical cooling units (nodes) 303 and supporting electrophysiology (EP) signal monitoring. Optical laser cooling may be used independently as a medical treatment method for cardiac arrhythmias. In one embodiment, the catheter integrates an optical laser cooling unit with intra-cardiac electrophysiological signal monitoring, mapping, processing and analysis. During an arrhythmia case (tissue ischemia or fibrillation), an EP catheter is used to track and localize an abnormal function and abnormal tissue of the heart. EP catheter 305 integrates optical cooling units 303 and traditional EP signal monitoring via signal sensors and enables real-time sensor signal analysis by analysis and control device 320 to identify abnormality. For example, the heart depicted in FIG. 3 includes abnormal functioning regions 331, 333, 335 and 337.

The signal mapping information and malfunction analysis results are used by EP signal analysis device 320 to provide a control signal to control laser optical cooling device controller 323 in real time. Signal analysis and control device 320 comprises a controller for controlling a laser optical signal provided to laser light emitting nodes 303 for optical cooling in response to a sensed temperature of anatomical tissue to regulate cooling of tissue. The device 320 controller controls the laser optical signal provided to said laser light emitting node by varying one or more of, laser pulse width, laser power and laser frequency. Thereby, laser optical cooling device controller 323 applies cooling treatment via cooling nodes 303 to abnormal functioning regions 331, 333, 335 and 337 with appropriate time duration and energy level. Catheter 305, control device 320 and cooling device 323 comprise a close loop computer processing system involving signal sensing, analysis and mapping to control laser optical cooling treatment, in response to pre-programmed instruction to advantageously treat cardiac arrhythmias. Catheter 305 senses, records and monitors heart electrophysiological activities using EP lead based signal mapping and analysis in conjunction with providing optical laser cooling via multiple laser cooling nodes. The optical laser cooling is usable in any kind of cardiac arrhythmia to manage temperature and thermal pattern control and steering. In another embodiment, two separate catheters are employed. A first catheter is used for EP signal monitoring and recording and a second catheter is used for laser cooling treatment.

In another embodiment, the tissue cooling, low temperature steering and thermal pattern control is achieved using magnetic cooling or electromagnetic cooling. With magnetic or electromagnetic cooling, the system provides low temperature non-invasive cooling. The non-invasive electromagnetic cooling advantageously decreases medical procedure complexity and risk, compared with invasive (non-optical) low temperature steering of an EP or other intra-cardiac catheters even though a degree of electromagnetic noise may be introduced.

In the optical laser cooling system embodiment, the system provides efficient and effective cooling for intra-cardiac treatment and procedures and is also usable for external devices to eliminate heat for skin and tissue treatment and surgery. The optical laser cooling system may be used in different kinds of surgeries to reduce heat and temperature in response to temperature sensor feedback and control by medical users. A user may control optical laser wave length, cooling duration, working mode (continuous or pulse), cooling pulse width and frequency, for example. This advantageously increases flexibility, safety and sensitivity in heat related medical procedures.

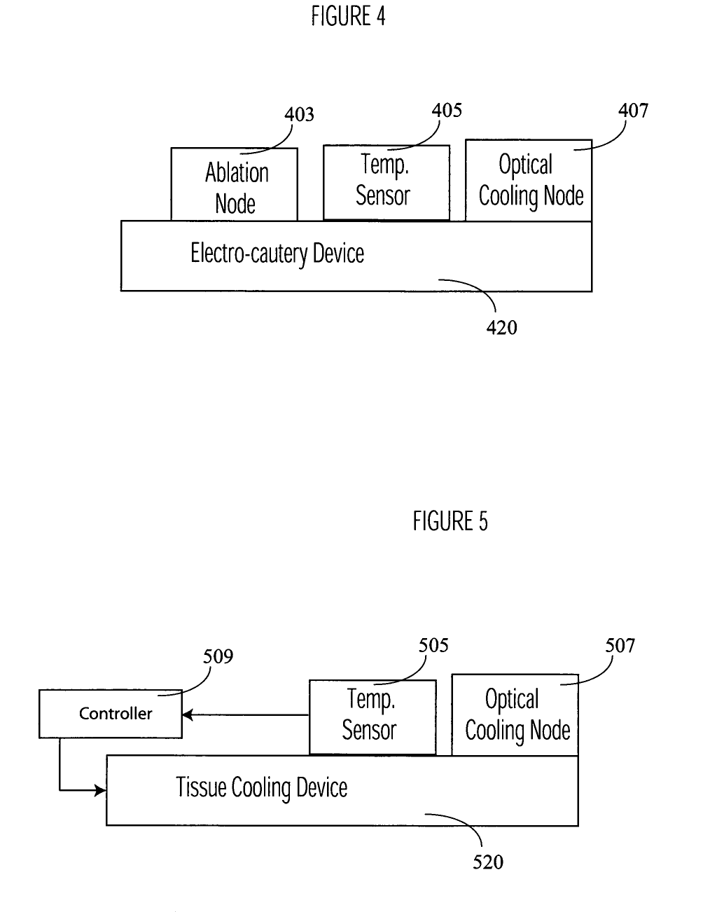

FIG. 4 shows an interventional electro-cautery system, comprising an electro-cautery device 420 for internal anatomical insertion. Electro-cautery device 420 includes a laser light emitting node 407 for optical cooling of anatomical tissue, and a laser optical (or RF) ablation node 403 for use in surgical removal of anatomical tissue. Temperature sensor 405 senses temperature of anatomical tissue for use in regulating at least one of, heating and cooling of tissue resulting from use of the ablation node and the laser light emitting node.

FIG. 5 shows an external anatomical tissue cooling system 520, comprising a laser light emitting node 507 for optical cooling of anatomical tissue. Temperature sensor 505 senses temperature of anatomical tissue for use in regulating cooling of tissue. Controller 509 controls a laser optical signal provided to laser light emitting node 507 for optical cooling in response to a sensed temperature from sensor 505 of anatomical tissue to regulate cooling of tissue.

The systems and functions of FIGS. 1-5 are not exclusive. Other systems and functions may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. An interventional system employs optical laser cooling (or magnetic or electromagnetic cooling in other embodiments) to manage temperature and thermal patterns in catheterization and other procedures. Further, functions and steps performed by the systems of FIGS. 1-5 may be implemented in hardware, software or a combination of both and may reside on one or more processing devices located at any location of a network or communication link linking the elements of FIGS. 1-5.

What is claimed is:

1. An interventional system provides tissue ablation and cooling, comprising:
a catheterization device for internal anatomical insertion including,
a laser light emitting node for optical cooling of anatomical tissue,
an ablation node for use in surgical removal of anatomical tissue and
a temperature sensor for sensing temperature of anatomical tissue for use in regulating at least one of, heating and cooling of tissue resulting from use of said ablation node and said laser light emitting node.

2. A system according to claim 1, including
a signal monitor for intra-cardiac electrophysiological signal monitoring, mapping and processing.

3. A system according to claim 1, wherein
said catheterization device includes a plurality of laser light emitting nodes for optical cooling of anatomical tissue,

4. A system according to claim 1, wherein
said ablation node is a Radio Frequency (RF) ablation node for use in surgical removal of anatomical tissue.

5. A system according to claim 1, wherein
said ablation node is a laser ablation node for use in surgical removal of anatomical tissue.

6. A system according to claim 1, including
a controller for controlling a laser optical signal provided to said laser light emitting node for optical cooling in response to a sensed temperature of anatomical tissue to regulate cooling of tissue.

7. A system according to claim 1, wherein
said controller controls said laser optical signal provided to said laser light emitting node by varying laser pulse width.

8. A system according to claim 1, wherein
said controller controls said laser optical signal provided to said laser light emitting node by varying laser power.

9. A system according to claim 1, wherein
said controller controls said laser optical signal provided to said laser light emitting node by varying laser frequency.

10. An interventional system provides tissue ablation and cooling, comprising:
an electro-cautery device for internal anatomical insertion including,
a laser light emitting node for optical cooling of anatomical tissue,
an ablation node for use in surgical removal of anatomical tissue and
a temperature sensor for sensing temperature of anatomical tissue for use in regulating at least one of, heating and cooling of tissue resulting from use of said ablation node and said laser light emitting node.

11. An external anatomical cooling system, comprising:
a laser light emitting node for optical cooling of anatomical tissue;
a temperature sensor for sensing temperature of anatomical tissue for use in regulating cooling of tissue; and
a controller for controlling a laser optical signal provided to said laser light emitting node for optical cooling in response to a sensed temperature of anatomical tissue to regulate cooling of tissue.
